(19) United States
(12) Patent Application Publication
(10) Pub. No.: US 2014/0107519 A1
(43) Pub. Date: Apr. 17, 2014

(54) APPARATUS FOR MEASURING BRAIN LOCAL ACTIVITY

(71) Applicant: BRAIN FUNCTIONS LABORATORY, INC., Yokohama-shi (JP)

(72) Inventors: Toshimitsu MUSHA, Yokohama (JP); Haruyasu MATSUZAKI, Yokohama (JP); Yukio KOSUGI, Yokohama (JP); Yoshio OKAMOTO, Narashino (JP); Takashi ASADA, Tsukuba (JP)

(73) Assignee: BRAIN FUNCTIONS LABORATORY, INC., Yokohama-shi (JP)

(21) Appl. No.: 13/650,864

(22) Filed: Oct. 12, 2012

(51) Int. Cl. A61B 5/048; A61B 5/00

(52) U.S. Cl. CPC A61B 5/048; A61B 5/7257
USPC 600/544

(57) ABSTRACT

A magnitude of a current component in x direction, y direction or z direction, or a composite current of the current components in x direction, y direction and z direction, estimated from scalp potentials outputted from sensors mounted on a head of a subject, predetermined coordinates of lattice points preset in a standard brain, and predetermined coordinates of the sensors is determined. A normalized power variance (NPV) and its mean value are determined with Fourier coefficients determined from the magnitude of the current component or the composite current. Z-score (or Y-score) of the subject from a mean value of NPV predetermined in the same manner as the mean value and a standard deviation of the NPV for a predetermined normal person group is determined and mapped with contour lines corresponding to the lattice points on a horizontal plane designated.

### BACKGROUND OF THE INVENTION

1. Field of the Invention

The present invention relates to an apparatus for measuring a brain local activity, and in particular to an apparatus for measuring or estimating a degree of neuronal impairment (diminishment) state in brain cortex such as a senile dementia disorder.

2. Description of the Related Art

With respect to senile dementia, it is statistically said that about 30% of nonagenarians are in dementia. This senile dementia is becoming a serious problem for the coming aging society.

Accordingly, such a dementia disorder should be preferably found out as early as possible and treated before it results in a serious state. As an apparatus for measuring (estimating) a degree of the dementia disorder, an apparatus for measuring a brain local activity has been already proposed. It comprises: a plurality of sensors mounted on a head of a subject for measuring scalp potentials or magnetic fields of the subject; a computing unit for converting alpha wave components of output signals of the sensors into numerical data to determine a dipolarity at each sampling, for determining mean values of squared errors, within a fixed time interval, between a scalp potential or a magnetic field by an equivalent dipole at a dipolarity peak emergence time and the measured scalp potentials or magnetic fields or variances of the squared errors from the mean values for the sensors, and for mapping a contour concerning a distribution of the mean values or the variances on a scalp or a brain surface corresponding thereto; and an output unit for outputting a contour map, as seen from e.g. Patent Document 1 (Japanese Patent No. 3,581,361).

The above-mentioned Patent Document 1 detects a functional impairment of neurons somewhere in a brain by analyzing a scalp potential distribution of alpha waves, so that there have been following problems:
a) There are a considerably high rate of persons (10-15%) not showing alpha waves;
b) The alpha waves are restrained in an eye-opening state, causing an extremely unstable measurement;
c) The alpha waves are highly affected by an emotional state;
d) Localization of a brain functional impairment degree only from the alpha waves does not properly coincide with a cerebral blood flow diminished part by SPECT (Single Photon Emission Computing Tomography).

Namely, since the Patent Document 1 consistently carries out a measurement based on the alpha waves, there have been problems that a functional impairment part of neurons can not be detected, a type and a degree of a brain disorder are unclear, and which part of the neuronal function has been recovered by various treatments is unclear.

Accordingly, some of the inventors of this patent application previously developed an apparatus for measuring a brain local activity by which a type and a degree of a brain disorder, and a part of a head where a neuronal function has been impaired or recovered can be specified without restrictions to the alpha waves, as seen from e.g. Patent Document 2 (Japanese Patent No. 4,145,344).

Namely, the above inventors of this patent application have discovered that when a neuronal function in brain cortex is impaired, neuronal activities become unstable; this influence emerges as a fluctuation of a local brain wave power (see T. Musha, T. Asada, F. Yamashita, T. Kinoshita, H. Matsuda, M. Uno, Z. Chen and W. R. Shankle, “A new EEG method for estimating cortical neuronal impairment that is sensitive to early stage Alzheimer's disease,” Clinical Neurophysiology, 113 (2002) 1052-1058); and this characteristic ranges over not only the area of the alpha waves but also the entire area of frequencies (e.g. 2-40 Hz) of brain waves wider than the alpha waves.

An apparatus for measuring a brain local activity developed based on the above art comprised: a plurality of sensors mounted on a head of a subject for measuring scalp potentials of the subject; and a computing unit dividing a predetermined frequency bandwidth wider than a frequency bandwidth of alpha waves of the scalp potentials outputted from the sensors into a predetermined number of frequency banks each having a fixed frequency bandwidth, dividing data of each divided frequency bank into segments of a predetermined duration on a time axis, determining a Z-score of the subject from a first mean value of normalized power variances (hereinafter NPV) determined for the segments and a second mean value of normalized power variances predetermined in the same manner as the first mean value for a predetermined normal person group and a standard deviation of the normalized power variances in the group, and mapping on a brain surface for each sensor a mean value of the Z-scores determined over all of the frequency banks. Thus, the map of the local neuronal function impairment is prepared.

This map indicates how far a neuronal activity of a subject deviates from that of a normal person, namely “abnormality” of the neuronal activity, for which a Z-score of the subject is calculated. There are two types of abnormality, in which if “Z-score>0”, it indicates that the fluctuation of the neuronal activity is larger than that of a normal person group, that is “unstable” while if “Z-score<0”, it indicates that the neuronal activity is “inactive” as compared with that of the normal person group.

As the entire brain waves from 2 Hz to 40 Hz are thus to be measured in order to avoid artifact such as blink and induction from a commercial AC power source, the frequency range applied can be enlarged than that of alpha waves, regardless of a condition of closed eyes or opened eyes.

Furthermore, if standard templates concerning various brain diseases are experimentally prepared, a differential (discrimination) diagnosis concerning those diseases can be performed. Moreover, details of curative effects for respective brain diseases can also be recognized from the change of the map.

However, the above Patent Document 2 has the following disadvantages:

1) The Z-score is only mapped on a brain surface, so that it is difficult to discriminate a brain disorder having a neuronal abnormal activity only different from the depth from the brain surface;

2) Upon calculating the normalized power variance (NPV), the brain potential, i.e. the time series data of the brain potential provided by the sensors (channels) on a scalp are performed with Fast Fourier Transform (FFT) to be divided into frequency banks, and with Inverse Fast Fourier transform (IFFT) to restore the time series data for the calculation of NPV, so that the execution of both the FFT calculation and IFFT calculation will accompany an excessive calculation time, causing a time delay. Therefore, if data processing is performed in a concentrated manner by a server through the Internet, a line congestion due to an increased number of system users is expected, thereby highly delaying the signal processing.

### SUMMARY OF THE INVENTION

It is accordingly an object of the present invention to provide an apparatus for measuring a brain local activity in which a three dimensional discrimination of neuronal activity abnormal region and the shortening of its possessing time are achieved.

The inventors of this patent application have found out that the value of NPV calculated per segment has a statistically large variation and so even after averaging over segments is made the S/N ratio is in the order of 1, which is caused by determining NPV per segment.

Accordingly, an apparatus for measuring a brain local activity according to the present invention comprises: a plurality of sensors mounted on a head of a subject for measuring scalp potentials of the subject; and a computing unit for determining a magnitude of a current component in x direction, y direction or z direction, or a composite current of the current components in x direction, y direction and z direction, estimated from scalp potentials outputted from the sensors, predetermined coordinates of lattice points preset in a standard brain, and predetermined coordinates of the sensors; dividing the magnitude of the current component or the composite current into segments of a predetermined duration on a time axis, determining Fourier coefficients after each segment is Fourier-transformed within a predetermined frequency range, determining mean squared values of absolute value over the segments for each of the Fourier coefficients, and forming frequency banks including a plurality of the mean squared values adjacent to each other; determining a normalized power variance with the mean squared value of absolute value of the Fourier coefficients adjacent to each other for each frequency bank and determining a mean value of the normalized power variance over all of the frequency banks; determining a Z-score of the subject from a mean value of normalized power variances predetermined in the same manner as the mean value of the normalized power variances and a standard deviation of the normalized power variances for a predetermined normal person group as a reference or determining a Y-score of the subject from the mean value of the normalized power variances as well as the standard deviation obtained from all of the lattice points of the subject instead of the normal person group; and mapping the Z-score or Y-score with contour lines corresponding to the lattice points on a horizontal plane designated.

The above predetermined frequency bandwidth is for example 2-40 Hz, the plurality adjacent to each other is for example 2 including 0.78 Hz, and the predetermined duration is for example 2.56 seconds.

Also, according to the present invention, the sensors can be set in a terminal device, the computing unit can be provided in a calculation center, and the terminal device and the calculation center can be connected through a communication line.

Also, the present invention can provide a non-transitory computer readable recording medium encoded with a computer program for measuring a brain local activity, the program when executed by a computer causes the computer to perform a method comprising: measuring scalp potentials for a subject from a plurality of sensors mounted on a head of the subject; and determining a magnitude of a current component in x direction, y direction, or z direction or a composite current of the current components in x direction, y direction, and z direction estimated from scalp potentials outputted from the sensors, predetermined coordinates of lattice points preset in a standard brain, and predetermined coordinates of the sensors; dividing the magnitude of the current component or the composite current into segments of a predetermined duration on a time axis, determining Fourier coefficients after each segment is Fourier-transformed within a predetermined frequency range, determining mean squared values of absolute value over the segments for each of the Fourier coefficients, and forming frequency banks including a plurality of the mean squared values adjacent to each other; determining a normalized power variance with the mean squared value of absolute value of the Fourier coefficients adjacent to each other for each frequency bank and determining a mean value of the normalized power variance over all of the frequency banks; determining a Z-score of the subject from a mean value of normalized power variances predetermined in the same manner as the mean value and a standard deviation of the normalized power variances for a predetermined normal person group as a reference or determining a Y-score of the subject from the mean value of the normalized power variances as well as the standard deviation obtained from all of the lattice points of the subject instead of the normal person group; and mapping the Z-score or Y-score with contour lines corresponding to the lattice points on a horizontal plane designated.

Thus, the present invention realizes displaying an abnormal position of neuronal activity within a brain, enabling a brain disorder diagnosis in an inexpensive, non-invasive, high sensitive and reliable manner, whereby it is expected to be popular among small medical facilities. This also serves as a suppression means for Alzheimer disease requiring an early detection for prevention and is also available as diagnosis and monitoring of a depression or a child with developmental disability having increased recently, so that the public role is large.

### BRIEF DESCRIPTION OF THE DRAWINGS

The above and other objects and advantages of the invention will be apparent upon consideration of the following detailed description, taken in conjunction with the accompanying drawings, in which the reference numerals refer to like parts throughout and in which:

FIG. 1 is a block diagram showing an arrangement [1] of an apparatus for measuring a brain local activity according to the present invention;

FIG. 2 is a block diagram showing an arrangement [2] of an apparatus for measuring a brain local activity through network according to the present invention;

FIG. 3 is a flowchart showing a mapping procedure of Z-score in an operation example 1 of an apparatus for measuring a brain local activity according to the present invention;

FIG. 4 is a flowchart more intelligibly showing the flowchart shown in FIG. 3;

FIG. 5 is a diagram showing an arrangement of grid or lattice points within a brain used in each operation example of an apparatus for measuring a brain local activity according to the present invention;

FIG. 6 is a diagram showing a contour line map of Z-score of x direction current component in each lattice point on a certain horizontal plane provided in the operation example 1 of an apparatus for measuring a brain local activity according to the present invention;

FIG. 7 is a diagram showing a contour line map of Z-score of y direction current component in each lattice point on a certain horizontal plane provided in the operation example 1 of an apparatus for measuring a brain local activity according to the present invention;

FIG. 8 is a diagram showing a contour line map of Z-score of z direction current component in each lattice point over a certain horizontal plane provided in the operation example 1 of an apparatus for measuring a brain local activity according to the present invention;

FIG. 9 is a flowchart showing a mapping procedure of Z-score determined with respect to a magnitude of current obtained by combining x, y, z direction current components in each lattice point on a certain horizontal plane provided in an operation example 2 of an apparatus for measuring a brain local activity according to the present invention;

FIG. 10 is a diagram showing a contour line map of Z-score determined with respect to a magnitude of current obtained by combining x, y, z direction current components in each lattice point on a certain horizontal plane provided in the operation example 2 of an apparatus for measuring a brain local activity according to the present invention; and

FIG. 11 is a flowchart showing a mapping procedure of Y-score in an operation example 3 of an apparatus for measuring a brain local activity according to the present invention.

### DESCRIPTION OF THE EMBODIMENTS

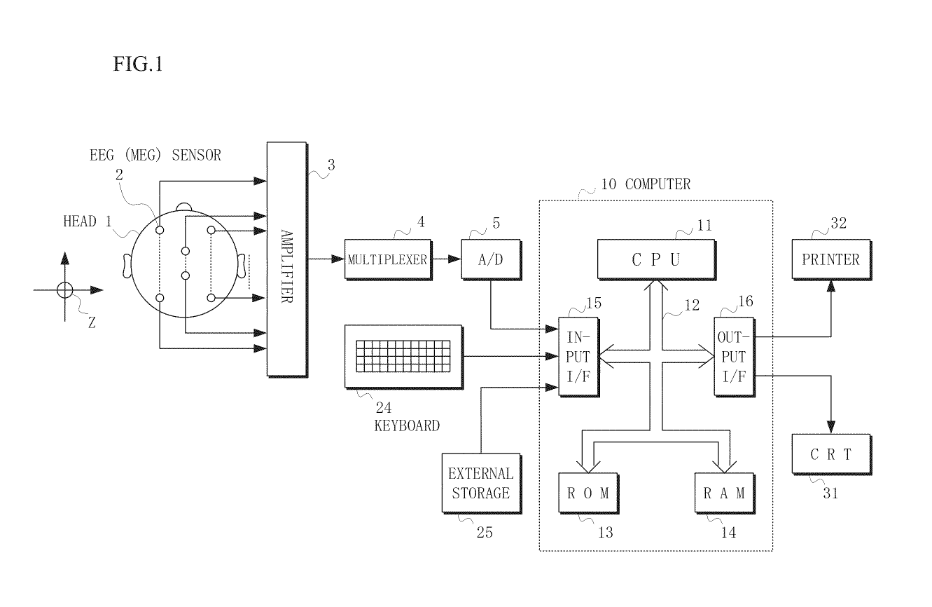

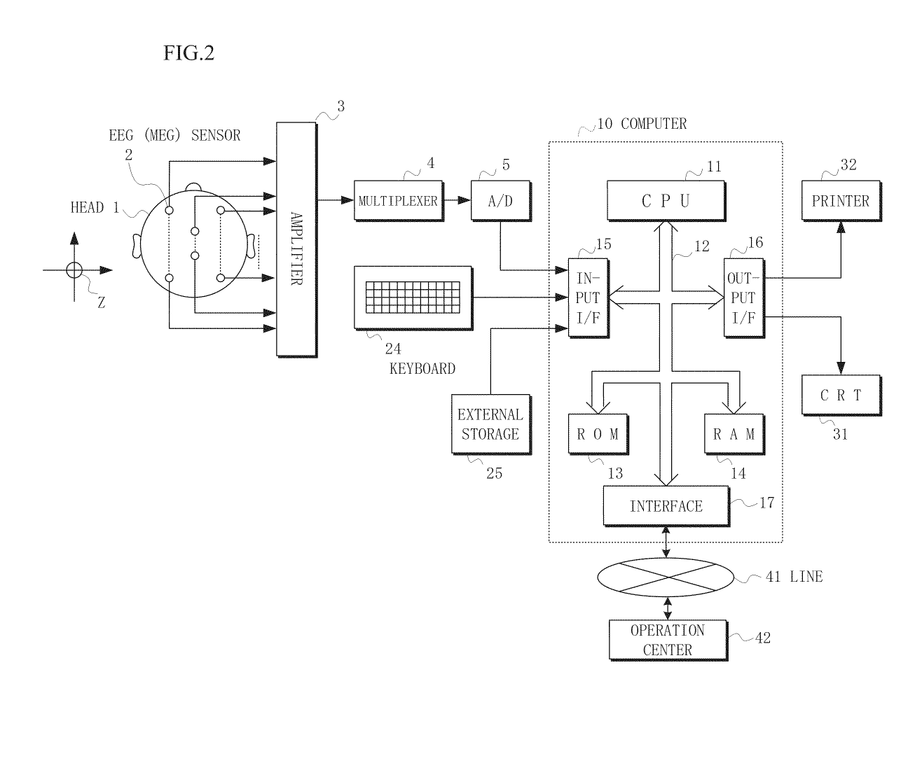

### Arrangement: FIGS. 1 and 2

FIG. 1 shows an arrangement [1] of an apparatus for measuring a brain local activity according to the present invention. In this arrangement, EEG sensors or MEG sensors both serving as electrodes (hereinafter occasionally referred to as sensors) 2₁-2₂₁ (hereinafter occasionally represented by a reference numeral 2) comprising e.g. around 21 sensors are firstly mounted on a head 1 to measure scalp potentials, or a subject puts on a cap or helmet where these sensors are properly arranged. It is to be noted that the sensors 2 in this case are arranged according to the international 10-20 standard, while for a reference potential, another sensor (not shown) is attached to e.g. a right ear lobe.

The scalp potential measured by the sensors 2 is supplied to an analog/digital (A/D) converter 5 through an amplifier 3 and a multiplexer 4, so that digitized measured potential (EEG) data is supplied to a computer 10 through an input interface (I/F) 15. It is to be noted that the input interface 15 may pass the data as it is or after only the component having a frequency bandwidth (e.g. predetermined frequency bandwidth wider than e.g. alpha wave) preliminarily designated is processed with digital filtering.

In the computer 10, a CPU 11 is connected to an ROM 13, an RAM 14, an input interface 15, and an output interface 16 through a bus 12. The above-mentioned ROM 13 is a read only storage medium, and the RAM 14 is a memory for storing EEG data from a keyboard 24 and the A/D converter 5 upon calculation.

Also, an external storage 25 for storing programs or the like is connected to the input interface 15. The display 31 of the CRT or the like which displays the operation result of the computer 10 and the printer 32 for printing the data and the waveform displayed at the display 31 are connected to the output interface 16 as output units. It is to be noted that all of the programs and the like may be stored only in the ROM 13 without using the external storage 25.

The above-mentioned brain wave data, as shown in an arrangement [2] of FIG. 2, is sent from an interface 17 of the computer 10 in a clinical site serving as a data transfer terminal equipment to an operation center 42, as a computing (arithmetic) unit through a communication line 41 of the Internet or the like, where the result analyzed at the operation center 42 is again sent back to the computer 10 through the communication line 41, and the result is outputted from an output unit such as a CRT 31 and a printer 32, so that the doctor may utilize the result as the materials for a diagnosis. In this case, a server, the program and the recording medium are provided in the operation center 42, in which data regarding brain potential are collected and accumulated at the server or the like, so that data regarding specific brain disorder is accumulated. By the computer 10, data per specific brain disorder may be referenced.

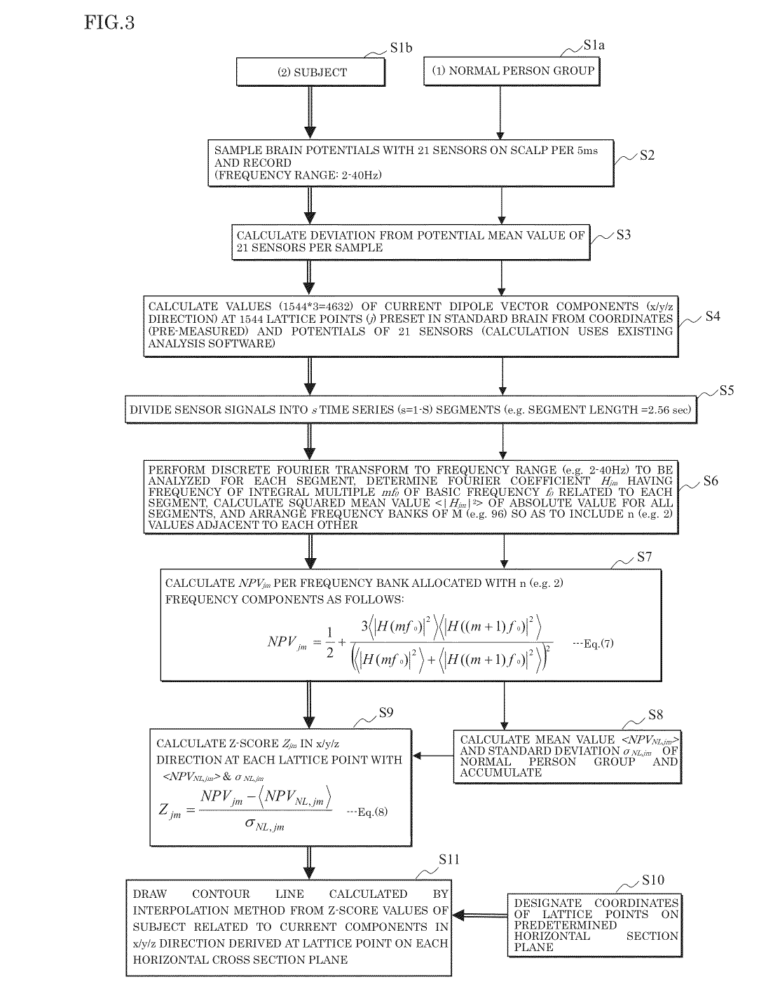

Embodiments of preparation of neuronal abnormality map and abnormality discrimination will now be described referring to FIGS. 3-11.

### *Operation Example 1

### Contour Line Map Example of Z-Score of x Direction (or y Direction or z Direction) Current Component at Each Lattice Point on a Horizontal Plane (See FIGS. 3-8)

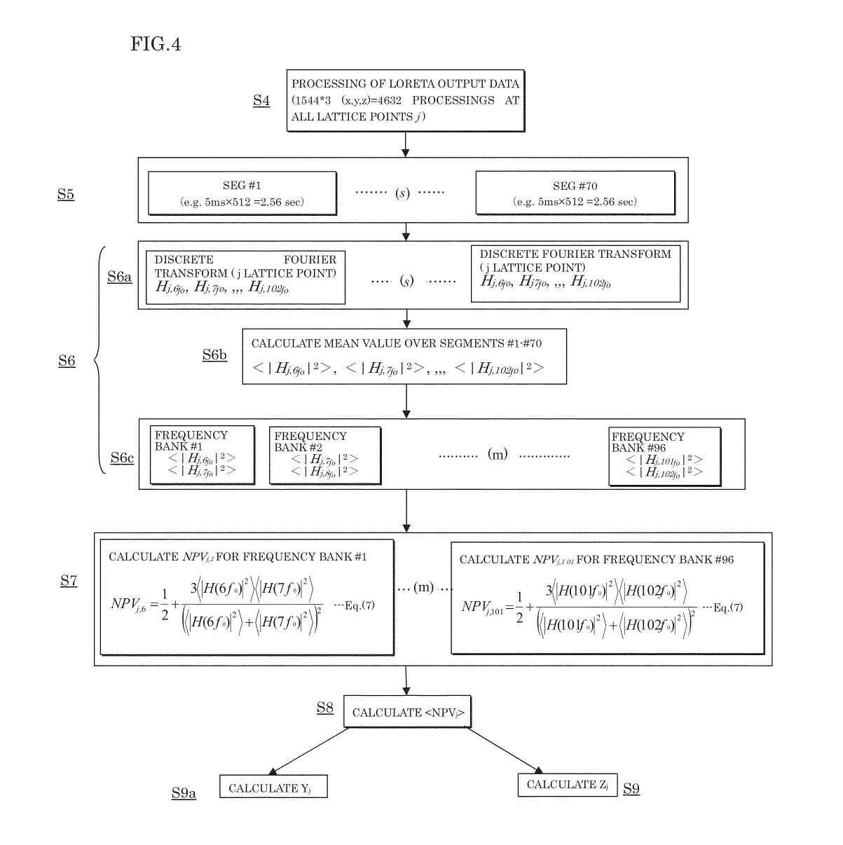

Operation example 1 of the above-mentioned arrangements will now be described along the flowcharts of FIG. 3 and FIG. 4, in which FIG. 4 specifically shows another aspect (processing part common to a subject, a normal person group and a group of brain disease patients (a group of persons of brain disorder)) of FIG. 3. It is to be noted that after the sensor group 2 is arranged on the head 1, the computer 10 is initialized upon power-up. Also, measuring the scalp potential based on the brain neuronal activity is performed at a fixed sampling time interval with the 21 sensors 2₁-2₂₁ mounted on the head 1.

### (1) Preparation of Database of Normal Person Group (Along Processing Route Shown by Solid Line Arrow)

### Step S1a, S2:

Firstly, a normal person group among a fixed number of persons is predetermined by the existing Mini-Mental State Examination (MMSE) method, the SPECT (Single Photon Emission Computing Tomography), or the like, where the scalp potentials on the head are measured one by one.

In this case, the potential (voltage) signals of the sensors 2₁-2₂₁ are sampled per 5 ms. The frequency region is a predetermined frequency band (e.g. 2-40 Hz), broader than that of a wave, which is set by a band pass filtering process.

### Step S3:

By subtracting a constant from each measurement so that the average of the 21 brain potentials for each sample may be zero, a deviation from the average of the brain potentials is calculated.

### Step S4:

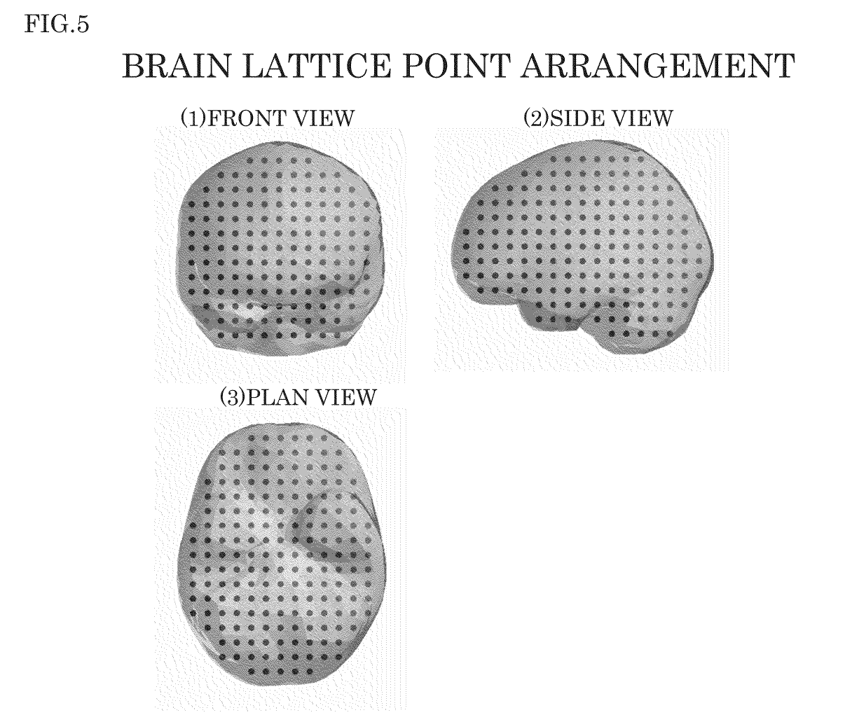

As shown in FIGS. 5A-5C, 1544 lattice points (j) are preset in a standard brain, in which head models of three layers (the standard brain is divided into three geometric areas of “skull”, “cerebral fluid”, and “brain tissue” with the electric conductivities of 1/80:3:1) are preliminarily arranged, so that cubic grids or lattices are assembled at 1 cm intervals within the standard head model and their position coordinates are determined where the number of lattice points is 1544. Values (1544*3=4632) of the vector components (x, y, or z direction) of a current dipole (hereinafter, referred to simply as current) at each lattice point are calculated from 21 sensor coordinates preset and the brain potentials measured of the sensors 2₁-2₂₁.

It is to be noted that the calculation method of a three dimensional electromotive force distribution in the brain from the potential distribution on a scalp, i.e. its algorithm is published as sLORETA, eLORETA (Low Resolution Brain Electromagnetic Tomography).

The processing of the LORETA output data is performed 1544*3 (x, y, z)=4632 times for all lattice points j.

### Step S5:

A signal of a sensor (channel) is divided into “s” (e.g. s=70) segments on a time axis, where each segment length is 2.56 seconds (=5 ms*512).

### Step S6:

Over the frequency range to be analyzed (e.g. 2-40 Hz), each segment is performed with discrete Fourier transform (step S6a) to obtain a Fourier coefficient Hⱼₘ having a frequency of an integral multiple mf₀ of the basic frequency f₀ for each segment (step S6a) and to calculate the mean squared value <|Hⱼₘ|²> of the absolute value over all segments (step S6b), thereby forming M (e.g. 96) frequency banks so as to include “n” (e.g. n=2) values adjacent to each other (step S6c).

Supposing that e.g. the sampling frequency is 200 Hz (5 ms) and the segment length is 2.56 seconds as above, the discrete frequency assumes an integral multiple of the basic frequency f₀ (=1/2.56=0.39 Hz), in which the lowest frequency bank #1 has 6f₀=2.34 Hz and 7f₀=2.73 Hz, the next bank has 7f₀ and 8f₀, . . . , the highest frequency bank #96 has 101f₀=39.41 Hz and 102f₀=39.8 Hz distributed, totaling 96 frequency banks divided.

### Step S7:

For each of the frequency banks #1-#96 thus allocated with two frequency components, the normalized power variance NPVⱼₘ upon designating the lattice point j and the frequency bank “m” (in this example, m starts from “6” where the frequency bank #1 corresponds to m=6) is calculated and stored in the RAM 14. The calculation of this NPVⱼₘ is performed in the following process.

xₙ (n=0−N−1) where a single segment of a length T sec includes N data samples is applied with Fourier expansion as follows, supposing that the Fourier coefficient is Hₘ and the frequency is mf₀ where f₀=1/T is the basic frequency:

$$\begin{matrix}{x}_{n} = \frac{1}{N} {\sum }_{m = 0}^{N - 1} {H}_{m} {W}_{N}^{nm} \\ where & Eq . ( 1 ) \\ {H}_{m} ≡ {\sum }_{n = 0}^{N - 1} {x}_{n } {e}^{- 2 π inm \text{/} N} ≡ {\sum }_{n = 0}^{N - 1} {x}_{n} {W}_{N}^{- nm} & Eq . ( 2 )\end{matrix}$$

In Eq. (1), when only two frequency components, i.e. mf₀ and (m+1)f₀ are left, n^{th} sample data is expressed as follows:

$${\overline{x}}_{n} = {x}_{n \text{:} m} + {x}_{n \text{:} m + 1} = \frac{1}{N} ( {H}_{m} {W}_{N}^{nm} + {H}_{m + 1} {W}_{N}^{n ( m + 1 )} + cc ) \qquad Eq . ( 3 )$$

where cc means a complex conjugate term.

By averaging over all of the segments, the following equation is obtained:

$$\begin{matrix}〈 {\overline{x}}_{n}^{2} 〉 = 〈 {( {x}_{n : m} + {x}_{n : m + 1} )}^{2} 〉 \\ = \frac{2}{{N}^{2}} ( 〈 {\vert {H}_{m} \vert}^{2} 〉 + 〈 {\vert {H}_{m + 1} \vert}^{2} 〉 ) .\end{matrix} \qquad Eq . ( 4 )$$

Similarly, the following equation is obtained:

[symbol]

x̅ₙ⁴ [symbol] = [symbol] x̅_{n:m}⁴ [symbol] + [symbol] x̅_{n:m+1} [symbol] +6( [symbol] x_{n:m}²x_{n:m+1}² [symbol] ) Eq. (5)

On the other hand, NPV is expressed by the following equation:

$$NPV = \frac{〈 {\overline{x}}_{n}^{4} 〉 - {〈 {\overline{x}}_{n}^{2} 〉}^{2}}{{〈 {\overline{x}}_{n}^{2} 〉}^{2}} \qquad Eq . ( 6 )$$

Therefore, by substituting Eqs. (4) and (5) for Eq. (6), the following Eq. (7) is obtained:

$${NPV}_{m} = \frac{1}{2} + \frac{3 〈 {\vert {H}_{m} \vert}^{2} 〉 〈 {\vert {H}_{m + 1} \vert}^{2} 〉}{{( 〈 {\vert {H}_{m} \vert}^{2} 〉 + 〈 {\vert {H}_{m + 1} \vert}^{2} 〉 )}^{2}} \qquad Eq . ( 7 )$$

### Step S8:

By the repetition of the above steps S1a-S7 for obtaining Eq. (7) for all of the normal persons, a group mean value <NPV_{NL, jm}> for the normal person group and the standard valuation σ_{NL, jm} inside the group are calculated and the result is stored in the RAM 14 as the database.

### (2) Preparation of Subject's Z-Score Map (Doubled Solid Line Arrow Route)

### Step S1b:

The scalp potentials from the sensors 2 equipped on the subject are measured.

### Steps S2-S7:

Steps S2-S7 are performed as with the case of the above normal person group to calculate the normalized power variance NPVⱼₘ of the subject.

### Step S9:

The Z-score of the subject is obtained from the normalized power variance NPVⱼₘ of the subject, the mean normalized power variance <NPV_{NL,jm}> of the normal person group, and its standard deviation σ_{NL, jm} in accordance with following equation:

$${Z}_{jm} = \frac{{NPV}_{jm} - {〈 NPVm 〉}_{NL}}{{σ}_{jm}} \qquad Eq . ( 8 )$$

### Step S10:

For a standard head model, the shape of the horizontal cross section plane spaced by 1 cm and the coordinates of the lattice points are designated so as to include the lattice points designated.

### Step S11:

From the values of the Z-scores of the subject related to the current of x direction, y direction or z direction derived at the lattice points on the horizontal cross section plane, contour lines are drawn by the calculation according to interpolation method.

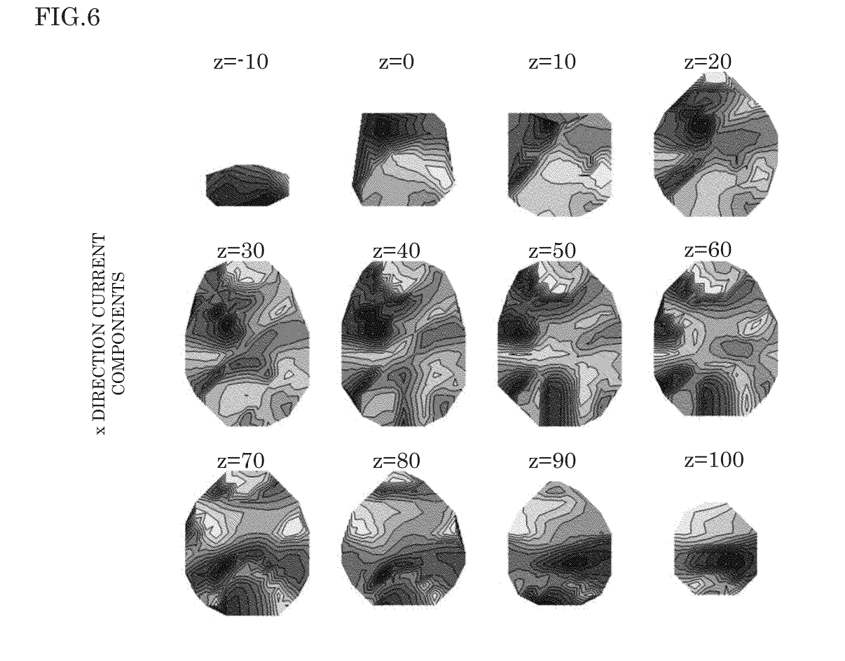

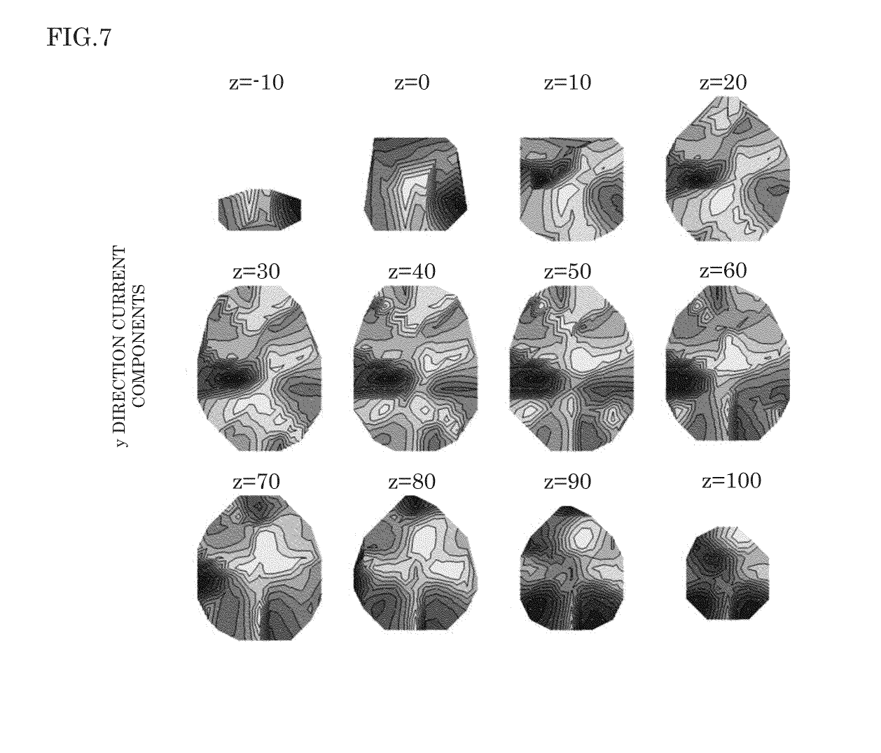

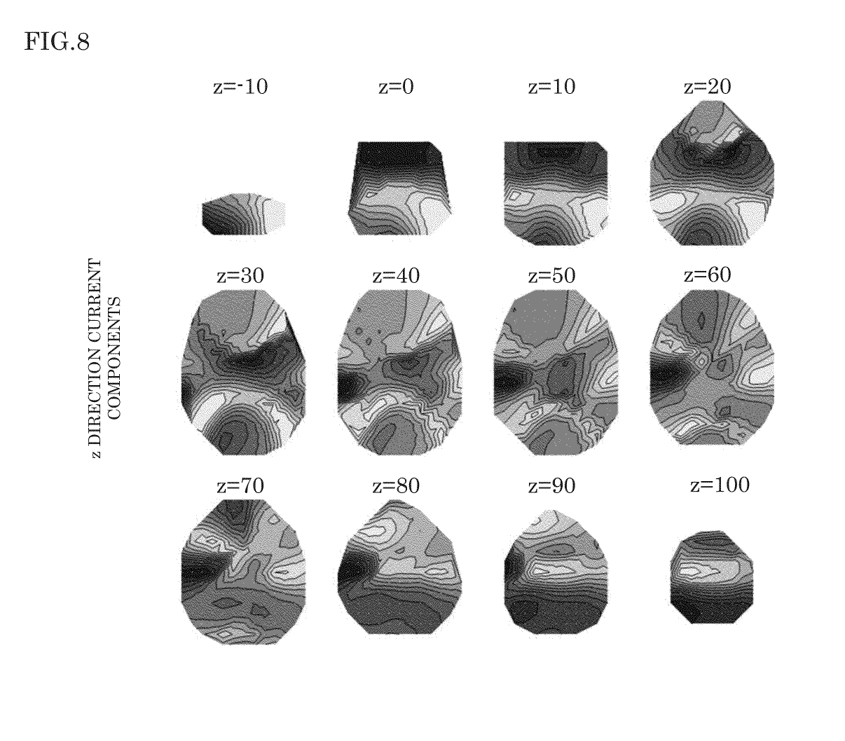

The contour lines in x direction are shown in FIG. 6, the contour lines in y direction are shown in FIG. 7, and the contour lines in z direction are shown in FIG. 8, where any of them shows the vertical direction position by z when slicing the brain in the horizontal planes.

These contour lines indicates that the positive (+) values of the Z-score becomes larger as the image figure becomes whiter while the negative (−) values of the Z-score becomes larger as it becomes blacker, indicating neuronal abnormal parts.

Namely, the case where Z-score assumes the minus value corresponds to a case where the normalized power variance NPVⱼ has a value smaller than the mean value <NPVⱼ> related to the lattice points j of the normal person group. Specifically, this indicates that the power variation at the lattice point of the subject is smaller than the power fluctuation at the corresponding lattice points j of the normal person, representing an abnormality of inactive neuronal activity compared with the normal persons.

Contrarily, the value of Z-score being plus indicates an abnormality as well where the fluctuation of neuronal activity is larger than that of the normal person group, that is an “unstable” state, and a worse abnormality as the absolute value becomes larger.

### <Description of Speeding Up the Calculation Process>

In a case where each segment includes only two Fourier coefficients H_{f1}, H_{f2} as shown at Step S6c in FIG. 4, the NPV for each segment is calculated as given by Eq. (7). In this case, the calculation time is reduced to approximately 1% as compared with the case where the two Fourier coefficients are changed on a time basis by IFFT.

On the other hand, according to the present invention, when the frequency bank includes two frequency components (mean value among segments of the squared absolute value of Fourier coefficient), the time series data during 5 minutes are divided into approximately 117 segments, enabling the NPV values to be directly calculated from the Fourier coefficients, so that the FFT calculation time is 117 without making IFFT calculations, thereby remarkably shortening the calculation time as compared with Patent Document 2 where the NPV calculation requires the IFFT calculation after the FFT calculation.

Furthermore according to the present invention, the squared value of the absolute value of Fourier coefficients is averaged over 70 segments and then the NPV is calculated by using the averaged value, so that the relative statistic noise included in NPV is reduced to (117)^{-0.5} times, that is 10%. On the other hand, the relative noise the Fourier coefficient has is 8 times the relative noise contained in the Fourier coefficient from the above Eq. (7), so that the relative noise contained in the NPV reaches to 2*8=16 times the noise contained in the Fourier coefficient, providing a high noise reduction effect.

### *Operation Example 2

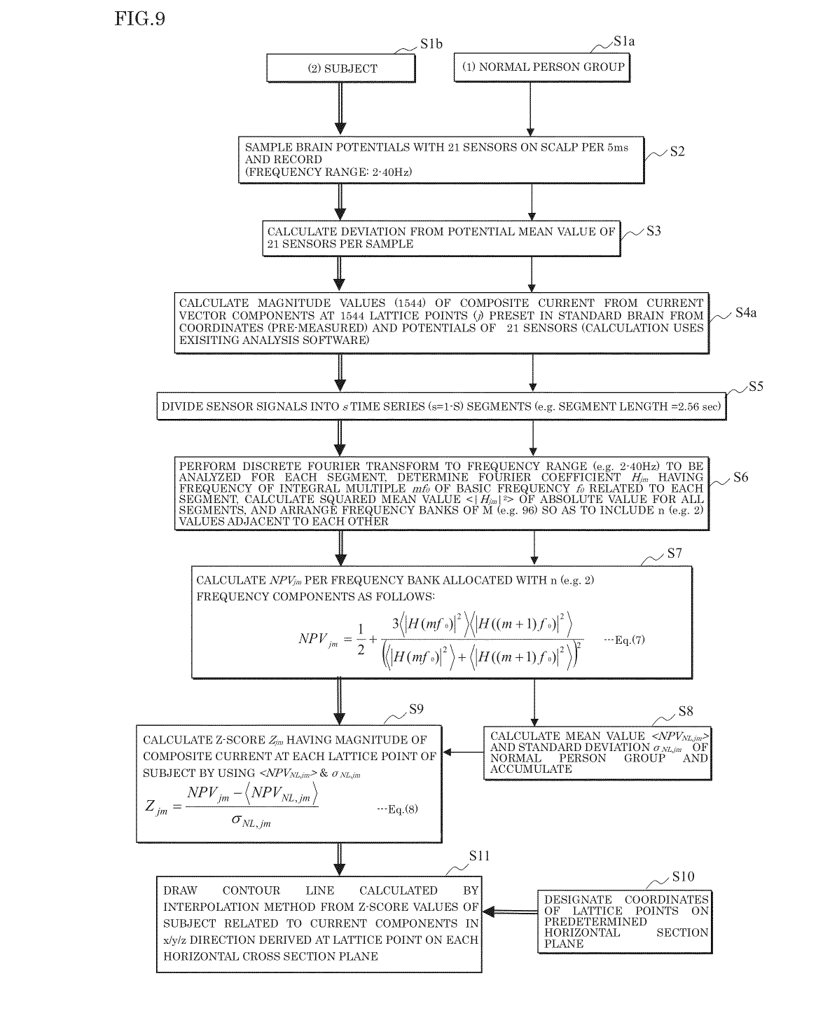

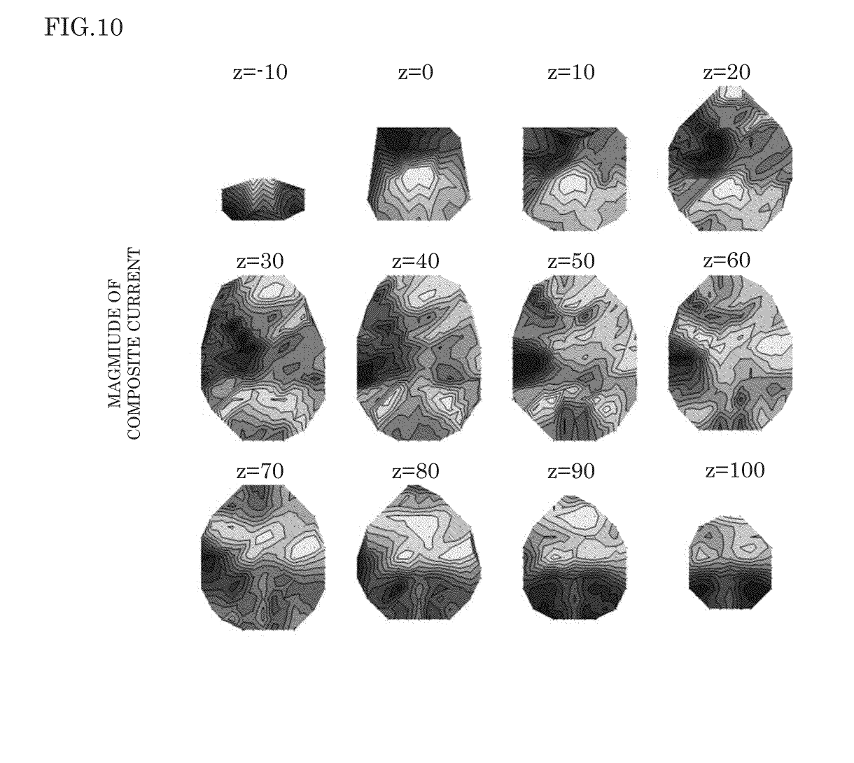

### Contour Line Mapping Example of Z-Score of Composite Current Magnitudes in x Direction, y Direction and z Direction at Each Lattice Point on a Horizontal Plane (See FIGS. 9 and 10)

The difference between this embodiment 2 and the above embodiment 1 is that step S4 in the flowchart of FIG. 3 is replaced by step S4a in FIG. 9.

Namely, while in case of embodiment 1, the contour line map shown in FIGS. 6-8 is obtained by the values of Z-score of a subject related to a current component in x direction, y direction or z direction derived at each lattice point on each horizontal plane, this embodiment 2 is different in that the contour line map is displayed by determining Z-score from the composite current magnitudes in x direction, y direction and z direction at each lattice point. Therefore, the output data processing of FIG. 4 is 1544 times, where the remaining is the same as embodiment 1.

### *Operation Example 3

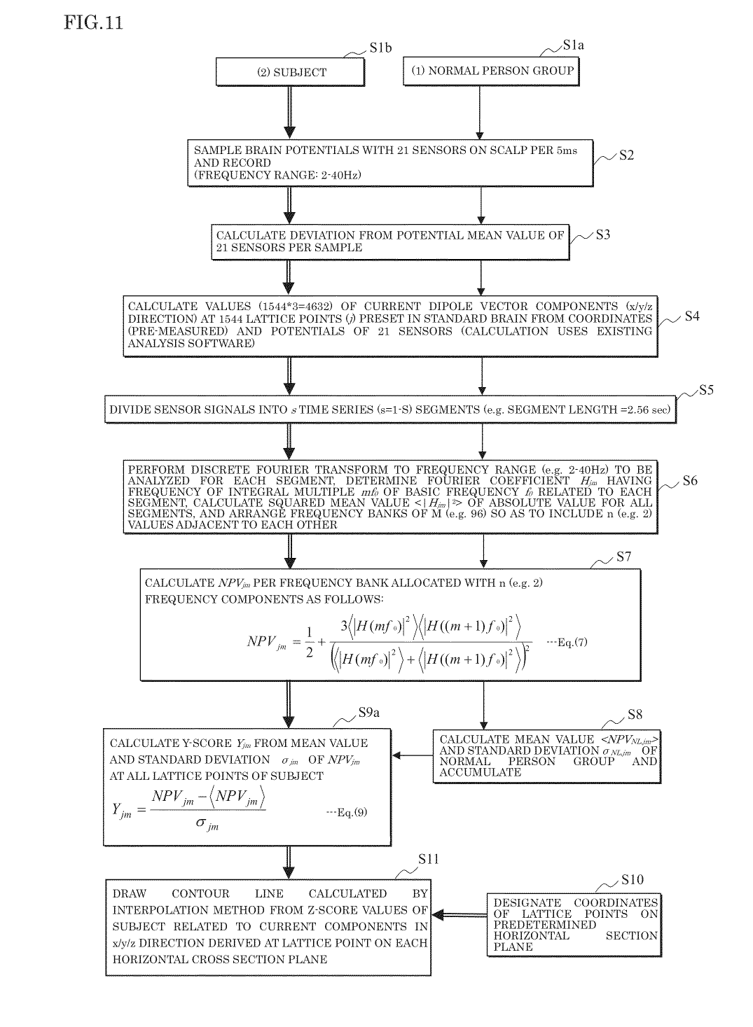

### Contour Line Mapping Example of Y-Score of x Direction (or y Direction or z Direction) Current Component at Each Lattice Point on a Horizontal Plane (See FIG. 11)

The difference between this embodiment and the above embodiment 1 is that step S9 in the flowchart FIG. 3 is replaced by step S9a in FIG. 11.

Namely, while in case of embodiment 1 the contour line map shown FIGS. 6-8 is obtained by the values of Z-score of a subject related to a current component in x direction, y direction or z direction derived at each lattice point on each horizontal plane, this embodiment 3 is different in that the contour line map is prepared by determining “Y-score” of the subject from the composite current magnitudes in x direction, y direction or z direction at each lattice point. The remaining is the same as embodiment 1, where the contour line map prepared is different from that of embodiment 1, the figures being omitted.

It is to be noted that Y-score Yⱼₘ in this embodiment is calculated from the mean value <NPVⱼₘ> of NPVⱼₘ at each lattice point and its standard deviation σⱼₘ in accordance with the following equation:

$${Y}_{jm} = \frac{{NPV}_{jm} - 〈 {NPV}_{jm} 〉}{{σ}_{jm}} \qquad Eq . ( 9 )$$

### *Operation Example 4

### Contour Line Mapping Example of Y-Score of Composite Current Magnitudes in x Direction, y Direction and z Direction at Each Lattice Point on a Horizontal Plane (not Shown)

The difference between this embodiment and the above embodiment 2 is that the preparation step S9 of Z-score in the flowchart of FIG. 9 is replaced by the preparation of contour line map by determining “Y-score” of a subject, where the contour line map is omitted to be shown.

It is to be noted that the present invention is not limited by the above mentioned embodiments, and it is obvious that various modifications to depression etc as brain disorder maybe made by one skilled in the art based on the recitation of the claims.

What is claimed is:

1. An apparatus for measuring a brain local activity comprising:
a plurality of sensors mounted on a head of a subject for measuring scalp potentials of the subject; and
a computing unit for determining a magnitude of a current component in x direction, y direction or z direction, or a composite current of the current components in x direction, y direction and z direction, estimated from scalp potentials outputted from the sensors, predetermined coordinates of lattice points preset in a standard brain, and predetermined coordinates of the sensors; dividing the magnitude of the current component or the composite current into segments of a predetermined duration on a time axis, determining Fourier coefficients after each segment is Fourier-transformed within a predetermined frequency range, determining mean squared values of absolute value over the segments for each of the Fourier coefficients, and forming frequency banks including a plurality of the mean squared values adjacent to each other; determining a normalized power variance with the mean squared value of absolute value of the Fourier coefficients adjacent to each other for each frequency bank and determining a mean value of the normalized power variance over all of the frequency banks; determining a Z-score of the subject from a mean value of normalized power variances predetermined in the same manner as the mean value of the normalized power variances and a standard deviation of the normalized power variances for a predetermined normal person group as a reference or determining a Y-score of the subject from the mean value of the normalized power variances as well as the standard deviation obtained from all of the lattice points of the subject instead of the normal person group; and mapping the Z-score or Y-score with contour lines corresponding to the lattice points on a horizontal plane designated.

2. The apparatus for measuring a brain local activity as claimed in claim 1, wherein the predetermined frequency bandwidth is 2-40 Hz, the plurality adjacent to each other is 2 including 0.78 Hz, and the predetermined duration is 2.56 seconds.

3. The apparatus for measuring a brain local activity as claimed in claim 1, wherein the sensors are set in a terminal device, the computing unit is provided in a calculation center, and the terminal device and the calculation center are connected through a communication line.

4. A non-transitory computer readable recording medium encoded with a computer program for measuring a brain local activity, the program when executed by a computer causes the computer to perform a method comprising:
measuring scalp potentials for a subject from a plurality of sensors mounted on a head of the subject; and
determining a magnitude of a current component in x direction, y direction, or z direction or a composite current of the current components in x direction, y direction, and z direction estimated from scalp potentials outputted from the sensors, predetermined coordinates of lattice points preset in a standard brain, and predetermined coordinates of the sensors; dividing the magnitude of the current component or the composite current into segments of a predetermined duration on a time axis, determining Fourier coefficients after each segment is Fourier-transformed within a predetermined frequency range, determining mean squared values of absolute value over the segments for each of the Fourier coefficients, and forming frequency banks including a plurality of the mean squared values adjacent to each other; determining a normalized power variance with the mean squared value of absolute value of the Fourier coefficients adjacent to each other for each frequency bank and determining a mean value of the normalized power variance over all of the frequency banks; determining a Z-score of the subject from a mean value of normalized power variances predetermined in the same manner as the mean value of the normalized power variances and a standard deviation of the normalized power variances for a predetermined normal person group as a reference or determining a Y-score of the subject from the mean value of the normalized power variances as well as the standard deviation obtained from all of the lattice points of the subject instead of the normal person group; and mapping the Z-score or Y-score with contour lines corresponding to the lattice points on a horizontal plane designated.
